(19) United States
(12) Patent Application Publication
(10) Pub. No.: US 2026/0327554 A1
(43) Pub. Date: Oct. 22, 2026

(54) METHOD, RECORDING MEDIUM AND ELECTRONIC DEVICE FOR PRODUCING MYCELIUM

(71) Applicant: CJ CHEILJEDANG CORPORATION, Seoul (KR)

(72) Inventors: Eun Hye KIM, Seoul (KR); Seung Yoon HWANG, Seoul (KR); Mi Ra SONG, Seoul (KR)

(73) Assignee: CJ CHEILJEDANG CORPORATION, Seoul (KR)

(21) Appl. No.: 19/115,587

(22) PCT Filed: Sep. 27, 2023
(86) PCT No.: PCT/KR2023/015030

§ 371 Date: Mar. 26, 2025

Related U.S. Application Data
(60) Provisional application No. 63/410,453, filed on Sep. 27, 2022.

(51) Int. Cl. C12N 1/14; A23L 31/00; A23L 31/10; C12N 1/18

(52) U.S. Cl. CPC C12N 1/14; A23L 31/00; A23L 31/10; C12N 1/18

(57) ABSTRACT

Provided is a technology for automatically determining an optimal fermentation method for producing mycelium based on the application of mycelium, the status of raw material inventory and supply and demand, the operating status and operating plan of production facilities, and the like.

### TECHNICAL FIELD

The present disclosure relates to a technology for producing mycelium. More specifically, the present disclosure relates to a technology for automatically determining the optimal fermentation (culture) method for producing mycelium based on the application of mycelium, the status of raw material inventory and supply and demand, the operating status and operating plan of production facilities, and the like.

### BACKGROUND ART

In response to social demands for environmentally-friendly materials, environmentally-friendly materials produced from various raw materials are being studied. As an example of such various raw materials, mycelium, which are composed of a lump of hyphae constituting the body of a mold, are attracting considerable attention. Since mycelium can be produced into various forms of intermediate products, it is expected that they will be applicable to various fields.

### DISCLOSURE

### Technical Problem

A technical problem to be solved through the present disclosure is to provide a technology for automatically determining an optimal fermentation method for producing mycelium.

Another technical problem to be solved through the present disclosure is to provide a technology for reducing the cost required for selecting a fermentation method of mycelium by using the relationship between the application, formulation, and fermentation method of mycelium.

Another technical problem to be solved through the present disclosure is to provide a technology for constructing an automated mycelium production system capable of flexibly and efficiently coping with small-scale production of a variety of mycelium species.

The technical problems of the present disclosure are not limited to the problems mentioned above, and other problems not mentioned will be clearly understood by those skilled in the art from the description below.

### Technical Solution

According to an embodiment of the present disclosure, a method performed by an electronic device may comprise steps of (i) determining one factor from a factor group comprising mycelium application factor, mycelium formulation factor, and mycelium fermentation method factor, and (ii) determining at least one undetermined factor from the factor group based on the factor determined in the step (i).

In an embodiment, the mycelium application factor may be at least one application selected from an application group comprising a packaging material, building material, food, pharmaceutical, textile, and leather substitute.

In an embodiment, the food may be a meat protein substitute, a milk protein substitute, a protein substitute for a beverage, or a flavor enhancer.

In an embodiment, the leather substitute may be a leather substitute for bags, a leather substitute for shoes, a leather substitute for furniture, a leather substitute for clothing, or a leather substitute for household goods.

In an embodiment, the mycelium formulation factor may be at least one formulation selected from a substrate particle-mycelium complex, mycelium foam, mycelium slurry, and mycelium mat.

In an embodiment, the mycelium fermentation method factor may be at least one fermentation method selected from a fermentation method group comprising solid-state fermentation, liquid-state fermentation, and solid-state and liquid-state mixed fermentation.

In an embodiment, the step (i) may comprise a step of transmitting first display information for visualizing and displaying the factor group to a user terminal.

In an embodiment, the step (i) may comprise a step of transmitting second display information for visualizing and displaying the factor determined in the step (i) to a user terminal.

In an embodiment, the step (ii) may comprise a step of transmitting third display information for visualizing and displaying the factor determined in the step (ii) to a user terminal.

In an embodiment, the step (ii) may comprise a step of transmitting fourth display information for visualizing and displaying the factor determined in the step (i) and the factor determined in the step (ii) on one screen to a user terminal.

In an embodiment, the step (ii) may comprise a step of determining at least one undetermined factor from the factor group based on attribute information of the factor determined in the step (i).

In an embodiment, the attribute information of the mycelium application factor may be information about at least one of density, hardness, strength, tensile strength, tear strength, elasticity, color, edibility, durability, flexural strength, functionality, flexibility, abrasion resistance, fire resistance, water resistance, hygroscopicity, coefficient of friction, biodegradability, plasticity, cushioning, formability, color development, surface adhesion, compressibility, resilience, mycelium species and chewability of the mycelium formulation required for an application.

In an embodiment, the attribute information of the mycelium formulation factor may be information about at least one of raw material inventory, raw material supply and demand, properties, density, strength, and mycelium species of the mycelium formulation.

In an embodiment, the attribute information of the mycelium fermentation method factor may be information about at least one of raw material inventory, raw material supply and demand, production scale, production rate, mycelium species, and operation status and operation plan of a production facility in the fermentation method.

In an embodiment, the method may further comprise a step of determining mycelium fermentation method factor different from a candidate mycelium fermentation method factor based on the attribute information of the factor determined in the step (i) or the attribute information of the factor determined in the step (ii) when it is determined that a production facility corresponding to the candidate mycelium fermentation method factor for determining the mycelium fermentation factor is unable to operate.

In an embodiment, the step of determining at least one undetermined factor from the factor group based on the attribute information of the factor determined in the step (i) may comprise a step of transmitting a fifth display information for visualizing and displaying the attribute information to a user terminal.

In an embodiment, the method may further comprise a step of transmitting a signal for the mycelium fermentation method factor to a production facility corresponding to the mycelium fermentation method factor.

In an embodiment, the factor determined in the step (i) may be the mycelium application factor, and the factor determined in the step (ii) may be the mycelium formulation factor or the mycelium fermentation method factor.

In an embodiment, the step (ii) may comprise a step of determining the mycelium formulation factor after determining the mycelium fermentation method factor.

In an embodiment, the step (ii) may comprise a step of determining the mycelium fermentation method factor after determining the mycelium formulation factor.

In an embodiment, the mycelium application factor may be packaging material or building material, the mycelium formulation factor may be a substrate particle-mycelium complex, and the mycelium fermentation method factor may be solid-state fermentation.

In an embodiment, the mycelium application factor may be food, pharmaceutical, textile, or leather substitute, the mycelium formulation factor may be mycelium foam, and the mycelium fermentation method factor may be solid-state fermentation.

In an embodiment, the mycelium application factor may be food, pharmaceutical, or a leather substitute, the mycelium formulation factor may be mycelium slurry, and the mycelium fermentation method factor may be liquid-state fermentation.

In an embodiment, the mycelium application factor may be food, pharmaceutical, or a leather substitute, the mycelium formulation factor may be mycelium mat, and the mycelium fermentation method factor may be solid-state and liquid-state mixed fermentation

In an embodiment, the factor determined in the step (i) may be the mycelium fermentation method factor, and the factor determined in the step (ii) may be the mycelium application factor or the mycelium formulation factor.

In an embodiment, the step (ii) may comprise a step of determining the mycelium formulation factor after determining the mycelium application factor.

In an embodiment, the step (ii) may comprise a step of determining the mycelium application factor after determining the mycelium formulation factor.

In an embodiment, the mycelium application factor may be packaging material or building material, the mycelium formulation factor may be a substrate particle-mycelium complex, and the mycelium fermentation method factor may be solid-state fermentation.

In an embodiment, the mycelium application factor may be food, pharmaceutical, textile, or leather substitute, the mycelium formulation factor may be mycelium foam, and the mycelium fermentation method factor may be solid-state fermentation.

In an embodiment, the mycelium application factor may be food, pharmaceutical, or a leather substitute, the mycelium formulation factor may be mycelium slurry, and the mycelium fermentation method factor may be liquid-state fermentation.

In an embodiment, the mycelium application factor may be food, pharmaceutical, or a leather substitute, the mycelium formulation factor may be mycelium mat, and the mycelium fermentation method factor may be solid-state and liquid-state mixed fermentation.

In an embodiment, the factor determined in the step (i) may be the mycelium formulation factor, and the factor determined in the step (ii) may be the mycelium application factor or the mycelium fermentation method factor.

In an embodiment, the step (ii) may comprise a step of determining the mycelium fermentation method factor after determining the mycelium application factor.

In an embodiment, the step (ii) may comprise a step of determining the mycelium application factor after determining the mycelium fermentation method factor.

In an embodiment, the mycelium application factor may be packaging material or building material, the mycelium formulation factor may be a substrate particle-mycelium complex, and the mycelium fermentation method factor may be solid-state fermentation.

In an embodiment, the mycelium application factor may be food, pharmaceutical, textile, or leather substitute, the mycelium formulation factor may be mycelium foam, and the mycelium fermentation method factor may be solid-state fermentation.

In an embodiment, the mycelium application factor may be food, pharmaceutical, or a leather substitute, the mycelium formulation factor may be mycelium slurry, and the mycelium fermentation method factor may be liquid-state fermentation.

In an embodiment, the mycelium application factor may be food, pharmaceutical, or leather substitute, the mycelium formulation factor may be mycelium mat, and the mycelium fermentation method factor may be solid-state and liquid-state mixed fermentation.

According to another embodiment of the present disclosure, there may be provided a non-transitory computer-readable recording medium having recorded a computer program to be executed by a processor, wherein the computer program is configured to allow the processor to perform one method of the above-described methods when the computer program is executed by the processor.

According to another embodiment of the present disclosure, an electronic device may comprise a communication interface configured to communicate with a network, a processor configured to execute a computer program comprising one or more instructions, and a memory storing the computer program, wherein the processor is configured to perform the method of any one of claims 1 to 38 when the computer program is executed by the processor.

### Advantageous Effects

According to the present disclosure, an optimal fermentation method for producing mycelium can be automatically determined.

According to the present disclosure, the cost required for selecting a fermentation method of mycelium can be reduced by using the relationship between the application, formulation, and fermentation method of mycelium.

According to the present disclosure, an automated mycelium production system capable of flexibly and efficiently coping with small-scale production of a variety of mycelium species can be constructed.

Effects according to the technical idea of the present disclosure are not limited to the effects mentioned above, and other effects not mentioned can be clearly understood by a person skilled in the art of the present disclosure from the description of the specification.

### BRIEF DESCRIPTION OF THE DRAWING

FIG. 1 illustrates an environment in which devices according to an embodiment of the present disclosure can be applied.

FIG. 2 illustrates a computing apparatus capable of implementing devices according to an embodiment of the present disclosure.

FIG. 3 is a flowchart illustrating a method according to an embodiment of the present disclosure.

FIG. 4 is a flowchart illustrating a method that may be referenced in various embodiments of the present disclosure.

FIG. 5 is a flowchart illustrating an example of the method illustrated in FIG. 3.

FIG. 6 illustrates a flowchart of a user interface that may be referenced in various embodiments of the present disclosure.

FIG. 7 illustrates a table illustrating the relationship among the applications, formulations and fermentation methods of mycelium that can be referenced in various embodiments of the present disclosure.

### BEST MODE FOR CARRYING OUT THE INVENTION

Various embodiments described in the present disclosure are exemplified for the purpose of clearly explaining the technical idea of the present disclosure, and are not intended to be limited to a specific embodiment. The technical idea of the present disclosure comprises various modifications, equivalents, alternatives, and optional combinations of all or some of the embodiments described in the present disclosure. In addition, the scope of the technical idea of the present disclosure is not limited to the various embodiments presented below or the specific description thereof.

The terms used in the present disclosure, comprising technical or scientific terms, unless otherwise defined, may have a meaning commonly understood by a person having ordinary knowledge in the technical field to which this disclosure belongs.

The expressions “includes,” “may include,” “comprises,” “may comprise,” “has,” and “may have” as used in this disclosure indicate the presence of targeted features (for example, functions, operations, or components), but do not exclude the presence of other additional features. That is, such expressions should be understood as open-ended terms that imply the possibility of comprising other embodiments.

The singular expressions used in the present disclosure, unless otherwise clearly defined in the context, may comprise plural meanings, and may be similarly applied to singular expressions set forth in the claims.

The expressions “1st,” “2nd,” or “first,” “second,” and the like as used in the present disclosure, unless otherwise clearly defined in the context, are used to distinguish one object from another in referring to a plurality of similar objects, and do not limit the order or importance among the objects.

The expressions “A, B, and C,” “A, B or C,” “at least one of A, B and C,” or “at least one of A, B or C” as used in the present disclosure may refer to listed items or all possible combinations of the listed items, respectively. For example, “at least one of A or B” may refer to (1) at least one A, (2) at least one B, and (3) at least one A and at least one B.

The expression “based on” as used in the present disclosure is used to describe one or more factors affecting a decision, act of judgment, or action described in a phrase or sentence comprising this expression, and this expression does not exclude additional factors affecting the decision, act of judgment, or action.

As used in the present disclosure, the expression that a component (for example, a first component) is “connected” or “linked” to another component (for example, a second component) may mean that the component is not only directly connected or linked to the other component, but also connected or linked thereto via a new other component (for example, a third component).

The expression “configured to” as used in the present disclosure may have the meanings of “set to do”, “having an ability to do”, “modified to do”, “made to do”, “capable of doing”, and the like depending on the context. This expression is not limited to the meaning of “specifically designed in hardware”, and for example, a processor configured to perform a particular operation may mean a general-purpose processor that can perform that particular operation by executing software, or a special-purpose computer that is structured through programming to perform that particular operation.

The term “mycelium” as used in the present disclosure may collectively refer to an aggregate derived from a strain. For example, the mycelium in the present disclosure may be derived from phylum Ascomycetes such as genus Fusarium, genus Aspergillus, genus Trichoderma, genus Rhizopus, genus Mucor, genus Neurospora, genus Cordyceps, genus Monascus, and genus Morchella, but the present disclosure is not limited thereto. For another example, the mycelium may be derived from phylum Basidiomycetes such as genus Ganoderma, genus Trametes, genus Agaricus, genus Pleurotus, genus Lentinula, and genus Grifola, but the present disclosure is not limited thereto.

The mycelium in the present disclosure may comprise, for example, Fusarium venenatum and Fusarium oxysporum of genus Fusarium, may comprise Aspergillus niger and Aspergillus oryzaevar of genus Aspergillus, may comprise Trichoderma reesei and Tricholoma matsutake of genus Trichoderma, may comprise Rhizopus oligosporus of genus Rhizopus, may comprise Neurospora crassa and Neurospora intermedia of genus Neurospora, may comprise Cordyceps sinensis and Cordyceps militaris of genus Cordyceps, may comprise Monascus purpureus of genus Monascus, or may comprise Morchella angusticeps, Morchella hortensis, golden Morchella, Morchella crassipes, Morchella hesculenta, and Morchella esculenta of genus Morchella. In addition, the mycelium may comprise, for example, Beauveria bassiana, Metarhizium anisopliae, Metarhizium flavoviride, Paecilomyces fumusoroseus, Gilbertella, Ophiocordyceps sinensis, Rasamsonia, Talaromyces, Penicillium, Acremonium, Humicola, Chaetomium, Rhizomucor, Paecilomyces, Aureobasidium, Chrysonillia, Phanerochaete, Trichaptum, Stereum, Phlebia, Laetiporus, Peniophora, Xylaria polymorpha, Xylaria hypoxylon, Xylaria filiformis, Xylaria longipes, Hirsutella, Ashersonia, Verticillium, Nomuraea, Thermomyces, Myceliophthora, Thermoascus, Thielavia, Stibella, Melanocarpus, Malbranchea, Dactylomyces, Canahomyces, Scytalidium, Myriococcum, Corynascus, and Coonemeria, which are derived from phylum Ascomycetes.

In addition, the mycelium may comprise, for example, Ganoderma lucidum, Ganoderma oregonense, Ganoderma tsugae, Ganoderma resinaceum, Ganoderma brownie, Ganoderma japonicum, Ganoderma applanatum, glossy Ganoderma, Ganoderma sessile and Ganodermataceae of genus Ganoderma, may comprise Trametes versicolor and Trametes hirsute of genus Trametes, may comprise Agaricus bisporus and Agaricus blazei of genus Agaricus, may comprise Pleurotus ostreatus, Pleurotus djamor, Pleurotus eryngii, Pleurotus citrinopileatus, Pleurotus sajorcoju, Pleurotus florida and Pleurotus digmer of genus Pleurotus, may comprise Lentinus edodes of genus Lentinula, or may comprise Grifola frondosa of genus Grifola. In addition, the mycelium may comprise, for example, Phellinus linteus, Agrocybe brasiliensi, Flammulina velutipes, Hypholoma capnoides, Hypholoma sublateriumm, Maitake, Agrocybe, Coprinus, Agrocybe aegerita, Coprinus comatus, Fomes fomentarius, Piptoporous betulinus, Tremella fuciformis, Pholiota microspore, Hericium erinaceus), Inonotus, Pycnoporus cinnabarinus, Sparassis crispa, Pine mushroom, Tuber melanosporum, Schizophyllum commune, Polyporus squamosus, Laetiporus sulphureus, Lepista nuda, Hypsizygus marmoreus, Albatrellus, Amillaria, Bondarzewia, Cantharellus, Cerioporus, Climacodon, Fistulina, Flammulina, Fomes, Fomitopsis, Fusarium, Herecium, Hydnum, Hypomyces, Hypsizygus, Ischnoderma, Laetiporus, Laricifomes, Lentinus, Lepista, Meripilus, Ophiocordyceps, Panelus, Piptoporus, Polyporus, Pycnoporellus, Rhizopus, Schizophyllum, Stropharia, Tuber, Tyromyces, Wolfiporia, Amanita porphyria, Auricularia auricula-judae, Boletus auripes, Tylopilus neofelleus, Inonotus obliquus, Sulfureus, Edulis, Mattirolomyces terfezioides, Phellinus, Perenniporia, Macrohyporia, Bjerkandera, Cerrena, Pisolithus tinctorius, acupuncture needle mushroom, Volvariel volvacea, Phellinus Phellinus igniarius, Salmoneostramineus, Esculenta, Tremella mesenterica, Polyporus Polyporous, Nameko, and Umbellatus, which are derived from phylum Basidiomycetes.

In addition, the mycelium may comprise, for example, Methylophilus, Arthrobacter, Acinetobacter, Pseudomonas, Bacillus, Streptomyces alboniger, Streptomyces natalensis, Streptomyces tendae, Propionibacterium freudenreichhii, Actinomucor elegans, Zymomonas mobilis, Actinomycetes, White-rot fungi, Zygomycota, Polyporaceae, Foitopsidaceae, Phycomyces blakesleeanus, Wooden shelf fungi, Soft-rot fungi, Brown-rot fungi, and Saccharomyces.

The term “user interface” as used in this disclosure may refer to a physical or virtual medium created to enable communication between a user and an object, system, machine, or computer program. According to the present disclosure, a user may check and/or select various information regarding the production of mycelium through a user interface provided to a user terminal from a control device that overall manages and controls the production of mycelium. Such a user interface will be described in detail later through a description of the specification.

Hereinafter, various embodiments described in the present disclosure will be described with reference to the attached drawings. In the attached drawings and descriptions of the drawings, identical or substantially equivalent components may be given the same reference numerals. Additionally, in the description of various embodiments below, redundant descriptions of identical or corresponding components may be omitted, but this does not mean that the components are not comprised in the embodiments.

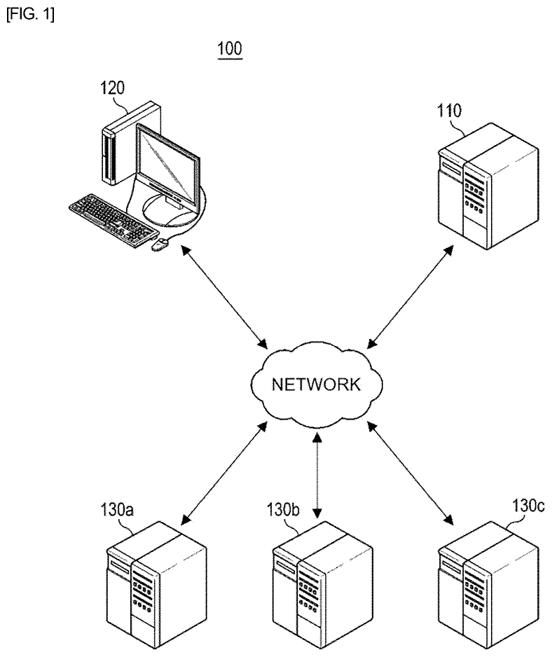

FIG. 1 illustrates an environment 100 to which devices 110, 120, 130a, 130b, and 130c according to an embodiment of the present disclosure can be applied. The environment 100 to be exemplarily explained through FIG. 1 is an environment in which a control device 110 overall manages and controls production facilities 130a, 130b, and 130c related to the production of mycelium, and transmits various information to a user terminal 120 through a user interface, or obtains responses to the various information from the user terminal 120.

Meanwhile, FIG. 1 illustrates an example in which one user terminal 120 and three production facilities 130a, 130b, and 130care each connected to a control device 110 via a network, but the number of user terminals 120 and production facilities 130a, 130b, and 130c may vary depending on the number of users or the number of production facilities. In addition, FIG. 1 only illustrates a preferred embodiment for achieving the purpose of the present disclosure, and some components may be merged into other components, some components may be deleted, or some components may be added, as needed. For example, the control device 110 and the production facilities 130a, 130b, and 130c may be implemented as distinct logic within a single device.

Hereinafter, each component illustrated in FIG. 1 will be described in detail.

The control device 110 may overall manage and control the production of mycelium. As a specific example of management and control performed by the control device 110, the control device 110 may determine the optimal fermentation method at least partially based on the application of mycelium, the status of raw material inventory and supply, the operating status and operating plan of production facilities, and the like. Here, the control device 110 may manage and control the production of mycelium by transmitting a signal regarding the optimal fermentation method to the production facilities 130a, 130b, and 130c corresponding to the method.

In addition, the control device 110 may transmit various information acquired, processed or produced during the process of producing mycelium to the user terminal 120 through a user interface. Here, various information transmitted to the user terminal 120 may comprise information requiring a user response (for example, user input). If a user response is obtained from the user terminal 120 through a user interface, the control device 110 may perform management and control regarding the production of mycelium based on the user response.

In addition to the operations of the control device 110 described so far, the control device 110 may interact with at least some of the user terminal 120 and production facilities 130a, 130b, and 130c to perform various operations for managing and controlling the production of mycelium. In addition, when producing a target product using production facilities, the control device 110 may be implemented by referring to known technologies in all technical fields for managing and controlling the production facilities. Hereinafter, with reference to the drawings in FIG. 3 and below, a technology for dynamically determining a fermentation method at least partially based on the use of mycelium, the status of raw material inventory and supply, the operating status and operating plan of production facilities, and the like as an operation performed by a control device 110, will be described in detail. However, it should be noted that this is not intended to exclude other operations performed by the control device 110 from the scope of the present disclosure.

The above-described control device 110 may be implemented with one or more computing devices. For example, all functions of the control device 110 may be implemented in a single computing device. For another example, the first function of the control device 110 may be implemented in a first computing device, and the second function thereof may be implemented in a second computing device. Such a computing device may be, for example, a desktop computer, a laptop computer, an application server, a proxy server, or a cloud server, but is not limited thereto, and any type of device equipped with computing functions may be a computing device.

The user terminal 120 may be a terminal used by a user. Here, the user may refer to a person who manages and controls the production of mycelium.

The user terminal 120 may receive various information acquired, processed or produced during the process of producing mycelium from the control device 110 and display the various information on the display of the user terminal 120. If the various information comprises information requiring a user response, the user terminal 120 may obtain a user input from the user and transmit the user input to the control device 110. Communication between the control device 110 and the user terminal 120 may be performed through a user interface.

The above-described user terminal 120 may be, for example, a desktop computer, a laptop computer, a tablet computer, a wearable device, or a smartphone, but is not limited thereto, and any type of device equipped with computing functions may be the user terminal 120. In addition, the user terminal 120 may be provided with a web browser or application to receive the above-described user interface from the control device 110.

The production facilities 130a, 130b, and 130c may be devices for culturing mycelium. Here, the production facilities 130a, 130b, and 130c may be implemented to correspond to a unique fermentation method. For example, the first production facility 130a may be a device for performing solid-state fermentation (SSF), the second production facility 130b may be a device for performing liquid-state fermentation (LSF), and the third production facility 130c may be a device for performing solid-state and liquid-state mixed fermentation. However, since this example corresponds to one production facility and one fermentation method for convenience of explanation, the scope of this disclosure is not limited to this example. In other words, unlike this example, one production facility may be implemented to correspond to a plurality of fermentation methods, or a plurality of production facilities may be implemented to correspond to one fermentation method.

The production facilities 130a, 130b, and 130c may start, stop, or control the production of mycelium based on a signal transmitted from the control device 110. Here, the signal transmitted from the control device 110 may comprise information about a fermentation method or information about control factors (for example, temperature, humidity, intensity, time, and the like) of the fermentation method.

Each of the above-described production facilities 130a, 130b, and 130c may be a device in which a specific device for producing mycelium is combined with a computing device. Such a computing device may be, for example, a desktop computer, a laptop computer, or an application server, but is not limited thereto, and any type of device equipped with computing functions may be a computing device.

Each of the devices (110, 120, 130a, 130b, 130c) illustrated in FIG. 1 described so far may communicate through a network. This network may be implemented as any type of wired or wireless network, such as, for example, a local area network (LAN), a wide area network (WAN), a mobile radio communication network (MRCN), or a wireless broadband (WiBro).

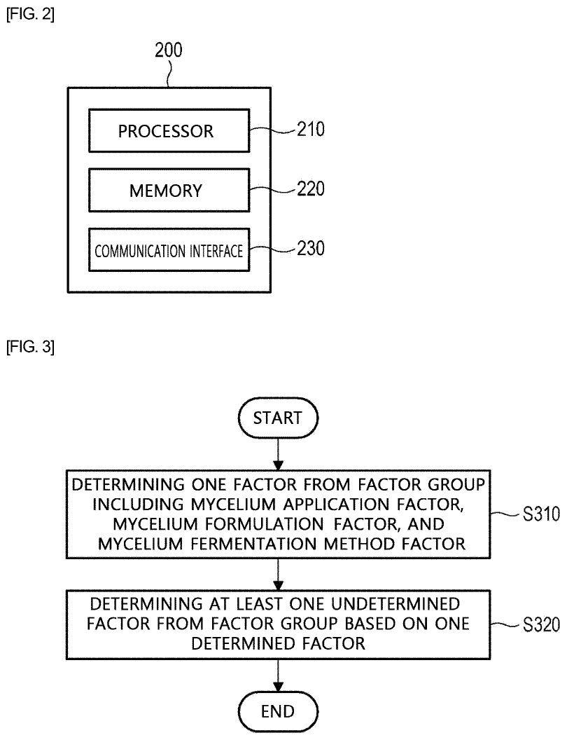

FIG. 2 illustrates a computing device 200 that can implement all or some of the devices 110, 120, 130a, 130b, and 130c according to an embodiment of the present disclosure. That is, all or some of the control device 110, user terminal 120, and production facilities 130a, 130b, and 130c illustrated in FIG. 1 may be implemented by the computing device 200 illustrated in FIG. 2. For reference, the computing device 200 in the present disclosure may be interchangeably referred to as an electronic device.

The computing device 200 may comprise at least one processor 210, at least one memory 220, or a communication interface 230. In an embodiment, some components may be removed from the computing device 200, or other components (for example, a display, an input device, and the like) may be added to the computing device 200. Additionally or alternatively, some of the components may be implemented in an integrated manner or may be implemented as a single or multiple entities. In the present disclosure, at least one processor 210 may be referred to as a processor 210. The term “processor 210” may be referred to as one processor or a collection of two or more processors, unless otherwise defined clearly. In addition, in the present disclosure, at least one memory 220 may be referred to as a memory 220. The term “memory 220” may be referred to as one memory or a collection of two or more memories, unless otherwise defined clearly.

Hereinafter, each component illustrated in FIG. 2 will be described in more detail.

The processor 210 may perform calculations or information processing related to the control or communication of each component of the computing device 200. Specifically, the processor 210 may control at least one component of the computing device 200 connected to the processor 210 by executing software (or a computer program) received from another component. As an example, the processor 210 may load a command (for example, an instruction, code, or code segment) or information into the memory 220, process the command or information stored in the memory 220, and store result information according to the processing in the memory 220. In addition, the processor 210 may be operatively connected to components of the computing device 200 to perform various operations, such as calculation, processing, and creation related to the present disclosure.

The memory 220 may store various information. The information stored in the memory 220 is information acquired, processed, or used by at least one component of the computing device 200, and may comprise software. The software may comprise one or more commands that, when loaded into the memory 220, allow the processor 210 to perform operations according to various embodiments of the present disclosure. That is, the processor 210 may perform operations according to various embodiments of the present disclosure by executing one or more of the commands described above. The memory 220 may comprise, for example, volatile or non-volatile memory. In an embodiment, the program, which is software stored in the memory 220, may comprise an operating system for controlling the resources of the computing device 200, an application, or middleware for providing various functions to the application so that the application can utilize the resources of the computing device 200.

The communication interface 230 may establish a wired or wireless communication channel with another device and transmit and receive various information to/from another device. In an embodiment, the communication interface 230 may comprise at least one port for connecting to another device via a wired cable to communicate with another device via wired communication. In this case, the communication interface 230 may communicate with another device connected by wire through at least one port. In an embodiment, the communication interface 230 may be configured to connect to a cellular network (for example, 3G, LTE, 5G, Wibro or Wimax) comprising a cellular communication module. In an embodiment, the communication interface 230 may comprise a near field communication module to transmit and receive information with other devices using near field communication (for example, Wi-Fi, Bluetooth, Bluetooth Low Energy (BLE), UWB). In an embodiment, the communication interface 230 may comprise a contactless communication module for contactless communication. The contactless communication may comprise at least one contactless proximity communication technology such as near field communication (NFC), radio frequency identification (RFID) communication, or magnetic secure transmission (MST) communication. In addition to the various examples described above, the computing device 200 may be implemented in various known ways to communicate with other devices, and the scope of the present disclosure is not limited by the examples described above.

In an embodiment, the computing device 200 may comprise a display. The display may display various screens (for example, one or more pages) based on the control of the processor 210. To display a screen to which various interfaces are applied, for example, a web browser or a dedicated application may be installed in the computing device 200. In addition, the display is a user-interactive configuration capable of receiving user input from a user. This display may be implemented in the form of a touch sensor panel (TSP) capable of recognizing the contact or proximity of various external objects (for example, a user's finger or stylus).

In an embodiment, the computing device 200 may comprise an input device (for example, a mouse or keyboard). The input device may receive information to be used in the components of the computing device 200 from an external source (for example, a user) of the computing device 200.

The processor 210, memory 220, and communication interface 230 illustrated in FIG. 2 may be connected to each other through a bus, general purpose input/output (GPIO), serial peripheral interface (SPI), or mobile industry processor interface (MIPI) to transmit or receive information or signals.

Hereinafter, methods according to various embodiments of the present disclosure will be described in detail. Although operations are illustrated in a particular order in the following drawings, it should be noted that operations do not necessarily have to be executed in a particular order or in a sequential order, or that all operations should be executed to achieve desired results.

In addition, the operations of methods to be described with reference to the following drawings may be performed by a computing device. In other words, the operations of the methods may be implemented by one or more instructions executed by a processor of a computing device. Although all of the operations comprised in such a method may be performed by one physical computing device, for example, a first operation of the method may be performed by a first computing device, and a second operation of the method may be performed by a second computing device. That is, the operations comprised in the method may be performed by dividing them into a plurality of computing devices.

Hereinafter, the explanation will continue assuming that the operation of the above-described method is performed by the control device 110 illustrated in FIG. 1. In addition, for convenience of explanation, a subject of the operation comprised in the method may be omitted, but unless otherwise defined in the context, it may be interpreted that the operation is performed by the control device 110.

FIG. 3 is a flowchart illustrating a method according to an embodiment of the present disclosure. The flowchart illustrated in FIG. 3 relates to a series of operations in which the control device 110 determines the application, formulation, and fermentation method of mycelium at least partially based on the application of mycelium, the status of raw material inventory and supply, the operating status and operating plan of production facilities, and transmits the determined optimal fermentation method to the corresponding production facility.

Hereinafter, the operations illustrated in FIG. 3 will be described in detail.

One factor may be determined from the factor group comprising mycelium application factor, mycelium formulation factor, and mycelium fermentation method factor (S310).

The mycelium application factor may refer to a purpose for which mycelium are used when the mycelium are produced through a series of processes. This mycelium application factor may be one or more applications selected from the application group comprising, for example, packaging materials, building materials, food, pharmaceuticals, textiles, and leather substitutes. Here, the application group may further comprise other applications in addition to the exemplified applications. Further, here, the food may be, for example, a meat protein replacer, a milk protein replacer, a protein replacer for a beverage, or a flavor enhancer. The mycelium capable of being used for such food may be, for example, genus Fusarium, genus Plutotus, genus Lentinula, genus Morcela, genus Agaricus, genus Monascus, genus Aspergillus, and genus Neurospora. Further, here, the leather substitute may be, for example, a leather substitute for bags, a leather substitute for shoes, a leather substitute for fumiture, a leather substitute for clothing or a leather substitute for household goods. As the mycelium capable of being used for such leather substitutes, for example, genus Ganoderma, genus Fusarium, genus Trametes, and genus Neurospora may be comprised.

The mycelium formulation factor may refer to a form of mycelium when the mycelium are produced through a series of processes. This mycelium formulation factor may be at least one formulation selected from a formulation group comprising a substrate particle-mycelium complex, mycelium foam, mycelium slurry, and mycelium mat. In particular, the substrate particle-mycelium complex may refer to solid-state formulation of a complex formed by the physical and/or chemical bonding of substrate particles and mycelium. In the substrate particle-mycelium complex, the substrate particle may be a nutrient for mycelium or may be a simple support that is not a nutrient. Further, in the substrate particle-mycelium complex, the substrate particles may be lignocellulose particles, fibrous particles, or the like.

Further, the mycelium foam may refer to solid-state formulation of mycelium having porosity by a directionless network. Further, the mycelium slurry may refer to liquid-state formulation comprising mycelium. Further, the mycelium mat may refer to solid-state formulation formed by mycelium with directionality. The above-described formulations may be interpreted as a broad concept comprising other formulations that can be treated as equivalent. Further, the formulation group may further comprise other formulations in addition to the exemplified formulations.

The mycelium fermentation method may be at least one fermentation method selected from a fermentation method group comprising solid-state fermentation, liquid-state fermentation, and solid-state and liquid-state mixed fermentation. Here, the solid-state fermentation may be a fermentation method that uses solid-state material as a substrate or support for culturing mycelium or that uses solid-state medium to fermentation mycelium. Further, the liquid-state fermentation may be a fermentation method comprising a process of stirring using a liquid medium or a process of using a bioreactor. As examples of such liquid-state fermentation, reference may be made to known technologies such as U.S. Patent Publication No. 455,485 entitled “Production of edible protein containing substances,” U.S. Patent Publication No. 478,2024 entitled “Fermentation process and fermenter,” and U.S. Patent Publication No. 556,3065 entitled “Controlling growth of filamentous microorganisms.” Further, the solid-state and liquid-state mixed fermentation may be a fermentation method that comprises both solid-state fermentation process and liquid-state fermentation process, or a fermentation method that uses a liquid medium without stirring (for example, liquid surface fermentation that fermentations mycelium on the surface of a liquid medium, or the like). The above-described fermentation methods may be interpreted as a broad concept comprising other fermentation methods that can be treated as equivalent. Further, the fermentation method group may further comprise other fermentation methods in addition to the exemplified fermentation methods.

The factor group may refer to a collection of factors whose determination is required in the production of mycelium. This factor group may comprise, for example, mycelium application factor, mycelium formulation factor, and mycelium fermentation method factor. Here, the factor group may further comprise other factors in addition to the exemplified factors.

This operation S310 may comprise an operation of determining a first target factor to be a target of a decision among the factors comprised in the factor group. For example, in the case where the factor group comprises mycelium application factor, mycelium formulation factor, and mycelium fermentation method factor, any one of these factors may be determined as a first target factor.

Regarding the operation of determining the first target factor, in an embodiment, the first target factor may be determined according to a predetermined priority. For example, when the mycelium application factor is determined in advance as a top priority, the mycelium application factor may be determined as the first target factor. Such a priority decision may be performed, for example, by an entity operating the control device 110.

Regarding the operation of determining the first target factor, in another embodiment, the first target factor may be determined randomly. For example, the mycelium formulation factor may be randomly determined as the first target factor.

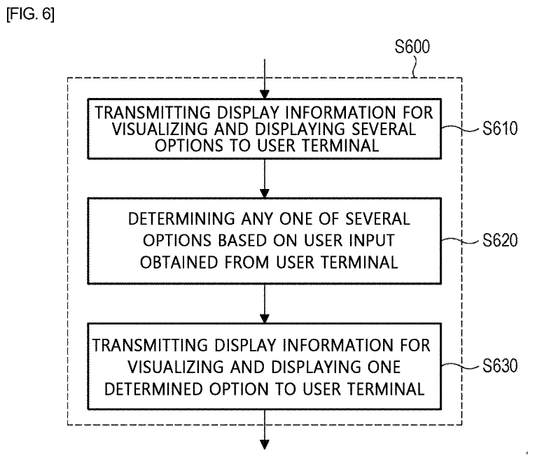

Regarding the operation of determining the first target factor, in another embodiment, the first target factor may be determined to correspond to a user input. For example, when mycelium fermentation method factor is selected by a user through a user interface, the mycelium fermentation method factor may be determined as the first target factor. That is, depending on a user's intention, any one of a plurality of options may be determined. To avoid redundant explanation, the operations for determining one of a plurality of options will be described later with reference to FIG. 6.

In addition, this operation S310 may comprise an operation of determining a value of a first target factor. For example, when mycelium application factor is determined as the first target factor, any one of packaging materials, building materials, food, pharmaceuticals, textiles, and leather substitutes as mycelium application factor may be determined as the value of the first target factor. As another example, when mycelium formulation factor is determined as the first target factor, any one of a substrate particle-mycelium complex, mycelium foam, mycelium slurry, and mycelium mat as mycelium formulation factor may be determined as the value of the first target factor. As another example, when mycelium fermentation method factor is determined as the first target factor, any one of solid-state fermentation, liquid-state fermentation, and solid-state and liquid-state mixed fermentation as mycelium fermentation method factor may be determined as the value of the first target factor.

Regarding the operation of determining the value of the first target factor, in an embodiment, the value of the first target factor may be determined according to a predetermined priority. For example, when mycelium application factor is determined as the first target factor and food is determined in advance as a top priority, the food may be determined as the value of the first target factor. As another example, when mycelium formulation factor is determined as the first target factor and mycelium foam is determined in advance as a top priority, the mycelium foam may be determined as the value of the first target factor. As another example, when mycelium fermentation method factor is determined as the first target factor and solid-state fermentation is determined in advance as a top priority, the solid-state fermentation may be determined as the value of the first target factor. Such a priority decision may be performed, for example, by an entity operating the control device 110.

Regarding the operation of determining the value of the first target factor, in another embodiment, the value of the first target factor may be determined based on a predetermined ratio. For example, when the first target factor is mycelium application factor and the predetermined ratio is {packaging material 10%, building material 10%, pharmaceutical 10%, food 30%, textile 30%, leather substitute 10%}, the value of the first target factor may be determined to correspond to that ratio. As another example, when the first target factor is mycelium formulation factor and the predetermined ratio is {10% substrate particle-mycelium complex, 30% mycelium foam, 30% mycelium slurry, 30% mycelium mat}, the value of the first target factor may be determined to correspond to that ratio. As another example, when the first target factor is mycelium fermentation method factor and the predetermined ratio is {solid-state fermentation 30%, liquid-state fermentation 30%, solid-state and liquid-state mixed fermentation 40%}, the value of the first target factor may be determined to correspond to that ratio. In order to determine the value of the first target factor so as to correspond to the predetermined ratio, each value may be determined sequentially (for example, packaging materials may be determined to produce 10% of the total production scale, and then building materials may be determined to produce 10% of the total production scale, etc.), each value may be determined in a mixed order (for example, substrate particle-mycelium complex, mycelium foam, mycelium slurry, and mycelium mat may be sequentially produced to produce 10% of the total production scale, and then mycelium foam may be determined to additionally produce 20% of the total production scale, etc.), or each value may be determined simultaneously and in parallel (for example, a process of determining a fermentation method corresponding to each value and transmitting a signal to production facilities may be performed in parallel). Such a ratio determination may be performed, for example, by an entity operating the control device 110.

Regarding the operation of determining the value of the first target factor, in another embodiment, the value of the first target factor may be determined randomly. For example, when mycelium application factor is determined as the first target factor, a leather substitute may be randomly determined as the value of the first target factor. As another example, when mycelium formulation factor is determined as the first target factor, mycelium slurry may be randomly determined as the value of the first target factor. As another example, when mycelium fermentation method factor is determined as the first target factor, liquid-state fermentation may be randomly determined as the value of the first target factor.

Regarding the operation of determining the value of the first target factor, in another embodiment, the value of the first target factor may be determined to correspond to a user input. For example, when mycelium application factor is determined as the first target factor and packaging material is selected by a user through a user interface, the packaging material may be determined as the value of the first target factor. As another example, when mycelium formulation factor is determined as the first target factor and mycelium mat is selected by a user through a user interface, the mycelium mat may be determined as the value of the first target factor. As another example, when mycelium fermentation method factor is determined as the first target factor and solid-state and liquid-state mixed fermentation is selected by a user through a user interface, the solid-state and liquid-state mixed fermentation may be determined as the value of the first target factor. That is, depending on a user's intention, any one of a plurality of options may be determined. To avoid redundant explanation, the operations for determining one of a plurality of options will be described later with reference to FIG. 6.

According to this operation S310, one factor (and value of the factor) comprised in the factor group may be determined. As will be specified in the contents of the specification later, there is a relationship among the mycelium application factor, the mycelium formulation factor, and the mycelium fermentation method factor, so that one factor comprised in the factor group may be determined, thereby determining a mycelium fermentation method corresponding to the determined factor.

Based on one determined factor, one or more other undetermined factors among the factor group may be determined (S320).

This operation S320 may comprise an operation of determining a second target factor to be a target of determination among the undetermined factors comprised in the factor group. For example, when the factor group comprises mycelium application factor, mycelium formulation factor, and mycelium fermentation method factor and the mycelium application factor is determined as the first target factor, any one of the mycelium formulation factor and the mycelium fermentation method factor may be determined as the second target factor. Regarding the operation of determining the second target factor, reference may be made to the description of the operation of determining the first target factor described above. In addition, this operation S320 may comprise an operation of determining a value of the second target factor. For example, when mycelium application factor is determined as the second target factor, any one of packaging materials, building materials, food, pharmaceuticals, textiles, and leather substitutes as mycelium application factor may be determined as the value of the second target factor. As another example, when mycelium formulation factor is determined as the second target factor, any one of a substrate particle-mycelium complex, mycelium foam, mycelium slurry, and mycelium mat as mycelium formulation factor may be determined as the value of the second target factor. As another example, when mycelium fermentation method factor is determined as the second target factor, any one of solid-state fermentation, liquid-state fermentation, and solid-state and liquid-state mixed fermentation as mycelium fermentation method factor may be determined as the value of the second target factor. Regarding the operation of determining the second target factor, reference may be made to the description of the operation of determining the value of the first target factor described above.

This operation S320 may comprise an operation of determining an undetermined third target factor comprised in the factor group after determining the second target factor. The operation of determining the third target factor may be understood as an operation that determines the last factor that has not been determined. In addition, this operation S320 may comprise an operation of determining a value of the third target factor. For example, when mycelium application factor is determined as the third target factor, any one of packaging materials, building materials, food, pharmaceuticals, textiles, and leather substitutes as mycelium application factor may be determined as the value of the third target factor. As another example, when mycelium formulation factor is determined as the third target factor, any one of a substrate particle-mycelium complex, mycelium foam, mycelium slurry, and mycelium mat as mycelium formulation factor may be determined as the value of the third target factor. As another example, when mycelium fermentation method factor is determined as the third target factor, any one of solid-state fermentation, liquid-state fermentation, and solid-state and liquid-state mixed fermentation as mycelium fermentation method factor may be determined as the value of the third target factor. Regarding the operation of determining the third target factor, reference may be made to the description of the operation of determining the value of the first target factor described above.

As an additional or alternative operation of this operation S320, the second target factor or the third target factor may be determined based on attribute information of the first target factor. Specifically, based on the attribute information of the value of the first target factor, the value of the second target factor or the value of the third target factor may be determined.

The attribute information of the first target factor may be, for example, attribute information of mycelium application factor, attribute information of mycelium formulation factor, or attribute information of mycelium fermentation method factor. Here, the attribute information of the mycelium application factor may be, for example, information about at least one of the density, hardness, strength, tensile strength, tear strength, elasticity, color, edibility, durability, flexural strength, functionality, flexibility, abrasion resistance, fire resistance, water resistance, hygroscopicity, coefficient of friction, biodegradability, plasticity, buffering, formability, color development, surface adhesion, compressibility, resilience, mycelium species, and chewability required for an application. Further, the attribute information of the mycelium formulation factor may be, for example, information about at least one of the density, strength, and mycelium species of the mycelium formulation. Further, the attribute information of the mycelium fermentation method factor may be, for example, information about at least one of the production scale and production speed, of the fermentation method, mycelium species, and the operating status and operating plan of production facilities.

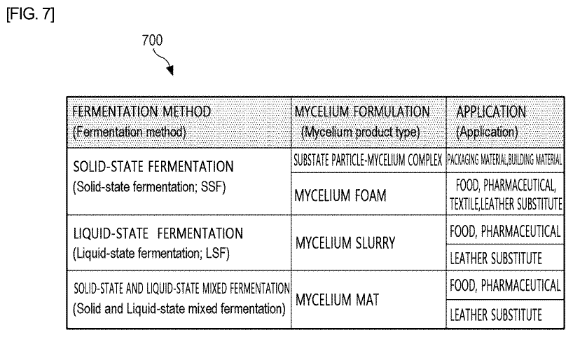

As a method of determination based on attribute information, for example, Table 700 shown in FIG. 7 may be referred to. This Table 700 may be a table that simply defines the relationship among the application, formulation, and fermentation method of mycelium by considering the attribute information of each factor comprised in the factor group.

Referring to Table 700 of FIG. 7, any pair of the application, formulation, and fermentation method of mycelium may have a one-to-one correspondence relationship. For example, {liquid-state fermentation} among the values of the mycelium fermentation method factor and {mycelium slurry} among the values of the mycelium formulation factor may have a one-to-one correspondence relationship. As another example, {solid-state and liquid-state mixed fermentation} among the values of the mycelium fermentation method factor and {mycelium mat} among the values of the mycelium formulation factor may have a one-to-one correspondence relationship.

In the case where there is a one-to-one correspondence relationship like this, the value of the target factor determined in a lower priority may be dynamically determined by the value of the target factor determined in a higher priority. For example, when mycelium formulation factor is determined as the first target factor, {mycelium slurry} is determined as its value, and mycelium fermentation method factor is determined as the second target factor (or the third target factor), {liquid-state fermentation} may be determined as the value of the second target factor (or the value of the third target factor). As another example, when mycelium fermentation method factor is determined as the second target factor, {solid-state and liquid-state mixed fermentation} is determined as its value, and mycelium formulation is determined as the third target factor, {mycelium mat} may be determined as the value of the third target factor.

Referring to Table 700 of FIG. 7, any pair of the application, formulation, and fermentation method of mycelium may have a one-to-many correspondence relationship. For example, {solid-state fermentation} among the values of the mycelium fermentation method factor and {substrate particle-mycelium complex} and {mycelium foam} among the values of the mycelium formulation factor may have a one-to-many correspondence relationship. As another example, {food} among the values of the mycelium application factor and {mycelium foam}, {mycelium slurry}, and {mycelium mat} among the values of the mycelium formulation factor may have a one-to-many correspondence relationship. As another example, {food} among the values of the mycelium application factor and {solid-state fermentation}, {liquid-state fermentation}, and {solid-state and liquid-state mixed fermentation} among the values of the mycelium fermentation method factor may have a one-to-many correspondence relationship.

In the case where there is a one-to-many correspondence relationship like this, the value of the target factor determined in a lower priority may be dynamically determined by the value of the target factor determined in a higher priority. The method of determining any one among a plurality of options may comprise a method of using a pre-determined priority, a method of using a pre-determined ratio, a method of using a user input, a method of using a random determination, or a method of using attribute information (for example, information about raw material inventory and supply, information about the operating status and operating plan of production facilities).

Regarding the method of using attribute information, as exemplified above, information about raw material inventory and supply may be used. Since unique raw materials may be required to produce each mycelium formulation, information about raw material inventory and supply may be additionally or alternatively considered to determine a value of one of the formulations comprised in the mycelium formulation factor. For example, when the value of the mycelium application factor, which is a target factor determined in a higher priority, is determined as {food}, the value of the mycelium formulation factor, which is a target factor determined in a lower priority, needs to be determined as a value of one of {mycelium foam}, {mycelium slurry}, and {mycelium mat}. Here, information on raw material inventory and supply may be additionally or alternatively taken into consideration, so that any one of {mycelium foam}, {mycelium slurry} and {mycelium mat} may be determined as a value of the mycelium formulation factor, which is a target factor determined in a lower priority.

Regarding the method of using attribute information, as exemplified above, information about the operating status and operating plan of production facilities may be used. Since the operating status and operating plan of each production facility need to be considered to perform each fermentation method, the operating status and operating plan of each production facility may be additionally or alternatively considered, and a value of one of the fermentation methods comprised in the mycelium fermentation method factor may be determined. For example, when the value of the mycelium application factor, which is a target factor determined as a higher priority, is determined as {food}, the value of the mycelium fermentation method factor, which is a target factor determined as a lower priority, needs to be determined as a value of one of {solid-state fermentation}, {liquid-state fermentation}, and {solid-state and liquid-state mixed fermentation}. In particular, information about the operating status and operating plan of production facilities may be additionally or alternatively taken into consideration, so that any one of {solid-state fermentation}, {liquid-state fermentation}, and {solid-state and liquid-state mixed fermentation} may be determined as a value of the mycelium fermentation method factor, which is a target factor determined as a lower priority.

Regarding the method of using attribute information, although not exemplified, it should be noted that any method of determining the value of a target factor determined as a lower priority by using attribute information of a target factor (or value of the target factor) determined as a higher priority is comprised in the scope of the present disclosure.

According to this operation S320, undetermined factors (and values of those factors) comprised in the factor group may be determined sequentially. All factors comprised in the factor group may be determined based on the relationship among mycelium application factor, mycelium formulation factor, and mycelium fermentation method factor, thereby determining an optimal mycelium fermentation method.

Additionally, a signal regarding the mycelium fermentation method factor may be transmitted to production facilities corresponding to the mycelium fermentation method factor (S330). This operation may be omitted depending on the implementation of an embodiment.

The determination of the mycelium fermentation method factor may be a result of the above-described operation S310 or operation S320.

In addition, as described above with reference to FIG. 1, when a unique fermentation method is determined for each of a plurality of production facilities, a signal regarding the determined fermentation method may be transmitted to the production facilities so as to correspond to the determined fermentation method.

According to various embodiments of the present disclosure described so far with reference to FIG. 3 and related exemplary drawings (for example, FIG. 7), an optimal mycelium fermentation method may be determined at least partially based on the application of mycelium, the status of raw material inventory and supply, the operating status and operating plan of production facilities, and the like. According to the automated determination of such a fermentation method, the production of mycelium in the production facilities corresponding to this fermentation method can be efficiently managed and controlled.

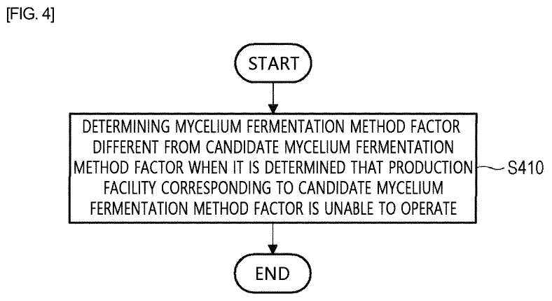

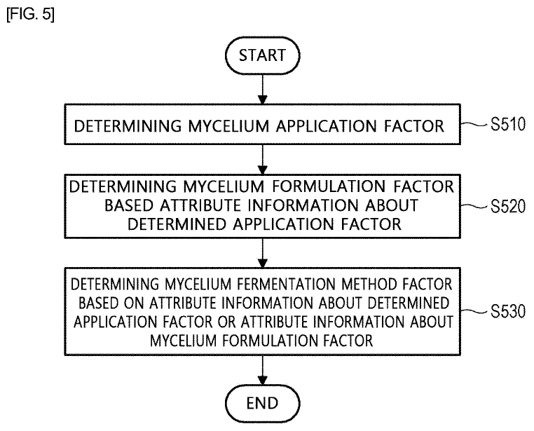

FIG. 4 is a flowchart illustrating a method that may be referenced in various embodiments of the present disclosure. The method illustrated in FIG. 4 comprises an operation for determining an optimal mycelium fermentation method in a situation where some production facilities are unable to operate. For example, the method illustrated in FIG. 4 may be applied in determining the mycelium fermentation method factor that may be comprised in the operation S310 or operation S320 illustrated in FIG. 3.

When it is determined that the production facility corresponding to a candidate mycelium fermentation method factor is unable to operate, mycelium fermentation method factor different from the candidate mycelium fermentation method factor may be determined (S410).

The candidate mycelium fermentation method factor may refer to a value that can become a candidate as mycelium fermentation method factor. For example, when there are {solid-state fermentation}, {liquid-state fermentation}, and {solid-state and liquid-state mixed fermentation} as values of the mycelium fermentation method factor, any one of them may be a candidate mycelium fermentation method factor.

According to this operation S410, when it is determined that the operation of the production facility corresponding to the candidate mycelium fermentation method factor is impossible, the value of the candidate mycelium fermentation method factor may be excluded, and the fermentation method factor may be determined with another value. In such exclusion, attribute information of a target factor (or attribute information of a value of a target factor) may be used.

For a specific example, when {solid-state fermentation} is determined as the candidate mycelium fermentation method factor, but the operation of production facilities corresponding to {solid-state fermentation} is impossible, {liquid-state fermentation} or {solid-state and liquid-state mixed fermentation} may be determined as a value of the mycelium fermentation method factor. In this determination, various operations for determining the values described with reference to FIG. 3 may be referred to. This determination operation S410 may be repeated until the value of the mycelium fermentation method factor can be determined, or may be stopped upon determining that it is impossible to determine all of the values of the mycelium fermentation method factor.

FIG. 5 is a flowchart illustrating an example of the method illustrated in FIG. 3. The method illustrated in FIG. 5 illustrates a series of operations in which mycelium application factor, mycelium formulation factor, and mycelium fermentation method factor are sequentially determined.

The mycelium application factor may be determined S510. The mycelium application factor may be determined as the first target factor. With regard to the determination of the target factor and the determination of the value of the target factor, refer to the description of FIG. 3.

The mycelium formulation factor may be determined based on attribute information about the determined application factor (S520). The mycelium formulation factor may be determined as the second target factor. Regarding the determination of the target factor and the determination of the value of the target factor, reference will be made to the description of FIG. 3. In particular, the description of the determination of the value of the target factor based on attribute information may be referred to in this operation.

The mycelium fermentation method factor may be determined based on attribute information about the determined application factor and attribute information about the determined mycelium formulation factor (S530). The mycelium fermentation method factor may be determined as the third target factor. Regarding the determination of the target factor and the determination of the value of the target factor, reference will be made to the description of FIG. 3. In particular, the description of the determination of the value of the target factor based on attribute information may be referred to in this operation.

Unlike the example illustrated in FIG. 5, target factors may be selected in any number of different orders. Specifically, the mycelium application factor, the mycelium fermentation method factor, and the mycelium formulation factor may be determined in sequence, the mycelium formulation factor, the mycelium application factor, and the mycelium fermentation method factor may be determined in sequence, the mycelium formulation factor, the mycelium fermentation method factor, and the mycelium application factor may be determined in sequence, the mycelium fermentation method factor, the mycelium application factor, and the mycelium formulation factor may be determined in sequence, or the mycelium fermentation method factor, the mycelium formulation factor, and the mycelium application factor may be determined in sequence. That is, factors may be selected sequentially in all possible combinations among the factors comprised in the factor group.

FIG. 6 is a flowchart of a user interface that may be referenced in various embodiments of the present disclosure. The operation S600 illustrated in FIG. 6 comprises a series of operations for managing and controlling the production of mycelium products based on a user's response. This operation S600 may be referenced in the determination operation of the target factor and the determination operation of the value of the target factor.

Hereinafter, the operations illustrated in FIG. 6 will be described in detail.

Display information for visualizing and displaying a plurality of options may be transmitted to the user terminal 120 (S610). In addition, based on a user input obtained from the user terminal 120, any one of the plurality of options may be determined (S620). In addition, display information for visualizing and displaying one of the determined options may be transmitted to the user terminal 120 (S630).

In the operation S310 of determining one factor from the factor group, for example, display information (for example, first display information) for visualizing and displaying the factor group may be transmitted to the user terminal 120, and based on a user input obtained from the user terminal 120, any one of the factors comprised in the factor group may be determined as the first target factor. Here, display information (for example, second display information) for visualizing and displaying the determined first target factor may be transmitted to the user terminal 120. The display information (for example, second display information) for visualizing and displaying the determined first target factor may comprise a value of the first target factor.

In the operation S320 of determining one or more other undetermined factors among the factor group, for example, display information for visualizing and displaying the undetermined factor comprised in the factor group may be transmitted to the user terminal 120, and a second target factor or a third target factor may be determined based on a user input obtained from the user terminal 120. Here, display information (for example, third display information) for visualizing and displaying the determined second target factor or third target factor may be transmitted to the user terminal 120. The display information (for example, third display information) for visualizing and displaying the determined second target factor or third target factor may comprise a value of the second target factor or a value of the third target factor.

In the operations S310 to S320 of determining the first to third target factors comprised in the factor group, display information (for example, fourth display information) for visualizing and displaying the determined first to third target factors on one screen may be transmitted to the user terminal 120. The display information (for example, fourth display information) for visualizing and displaying the determined first to third target factors may comprise a value of the first target factor to a value of the third target factor.

In the operations S310 to S320 of determining the first to third target factors comprised in the factor group, when attribute information is referenced, display information (for example, fifth display information) for visualizing and displaying the attribute information may be transmitted to the user terminal 120.

The determination of a plurality of options as above may or may not be optionally applied to at least some of the determination operations S310 to S320 illustrated in FIG. 3.

According to the embodiment illustrated in FIG. 6, a user's intention can be projected into the automated determination of the fermentation method. That is, according to the embodiment illustrated in FIG. 6, the mycelium application factor, mycelium formulation factor, and/or mycelium fermentation method factor that match the user's intention may be determined.

Heretofore, in the flowchart of the present disclosure, the operations of the method or algorithm have been described in a sequential order, but in addition to the operations performed sequentially, other operations may also be performed in an order that can be arbitrarily combined. The description of the flowchart of the present disclosure does not exclude changes or modifications to the method or algorithm, and does not imply that any operation is required or desirable. In an embodiment, at least some of the operations may be performed in parallel, iteratively, or heuristically. In another embodiment, at least some of the operations may be omitted, or other operations may be added.

Various embodiments of the present disclosure may be implemented as software in a machine-readable storage medium (MRSM) that can be read by a computing device. Software may be software for implementing various embodiments of the present disclosure. Software may be inferred from various embodiments of the present disclosure by programmers in the technical field to which the present disclosure belongs. For example, software may be a computer program comprising instructions that can be read by a computing device. The computing device is a device that can operate according to instructions called from a storage medium, and may be interchangeably referred to as an electronic device. In an embodiment, the processor of the computing device may execute the called instructions, thereby causing components of the computing device to perform functions corresponding to the instructions. The storage medium may refer to any type of recording medium that stores information that can be read by a device. The storage medium may comprise, for example, ROM, RAM, CD-ROM, a magnetic tape, a floppy disk, or an optical information storage device. In an embodiment, the storage medium may be implemented in the form of being distributed in a network-connected computer system. In this case, the software may be distributed, stored, and executed in a computer system, etc. In another embodiment, the storage medium may be a non-transitory storage medium. The non-transitory storage medium is a medium that exists regardless of whether information is stored semi-permanently or temporarily, and does not comprise signals that are transmitted transitorily.

Although the technical idea according to the present disclosure has been described through various embodiments above, the technical idea according to the present disclosure comprises various substitutions, modifications, and changes that can be made within a scope understandable to a person having ordinary skill in the art to which the present disclosure belongs. It should also be understood that such substitutions, modifications and alterations may be comprised within the scope of the appended claims.

1. A method performed by an electronic device, the method comprising steps of:
(i) determining one factor from a factor group comprising mycelium application factor, mycelium formulation factor, and mycelium fermentation method factor;
(ii) determining at least one undetermined factor from the factor group based on the factor determined in the step (i); and
(iii) controlling an operation of a production facility based on determination of the one factor and the at least one undetermined factor.

2. The method of claim 1,
wherein the mycelium application factor is at least one application selected from an application group comprising packaging material, building material, food, pharmaceutical, textile, and leather substitute.

3. The method of claim 2,
wherein the food is a meat protein substitute, a milk protein substitute, a protein substitute for a beverage, or a flavor enhancer.

4. The method of claim 2,
wherein the leather substitute is a leather substitute for bags, a leather substitute for shoes, a leather substitute for furniture, a leather substitute for clothing, or a leather substitute for household goods.

5. The method of claim 1,
wherein the mycelium formulation factor is at least one formulation selected from a substrate particle-mycelium complex, mycelium foam, mycelium slurry, and mycelium mat.

6. The method of claim 1,
wherein the mycelium fermentation method factor is at least one fermentation method selected from a fermentation method group comprising solid-state fermentation, liquid-state fermentation, and solid-state and liquid-state mixed fermentation.

7. The method of claim 1,
wherein the step (i) comprises a step of transmitting first display information for visualizing and displaying the factor group to a user terminal.

8. The method of claim 1,
wherein the step (i) comprises a step of transmitting second display information for visualizing and displaying the factor determined in the step (i) to a user terminal.

9. The method of claim 1,
wherein the step (ii) comprises a step of transmitting third display information for visualizing and displaying the factor determined in the step (ii) to a user terminal.

10. The method of claim 1,
wherein the step (ii) comprises a step of transmitting fourth display information for visualizing and displaying the factor determined in the step (i) and the factor determined in the step (ii) on one screen to a user terminal.

11. The method of claim 1,
wherein the step (ii) comprises a step of determining at least one undetermined factor from the factor group based on attribute information of the factor determined in the step (i).

12. The method of claim 11,
wherein the attribute information of the mycelium application factor is information about at least one of density, hardness, strength, tensile strength, tear strength, elasticity, color, edibility, durability, flexural strength, functionality, flexibility, abrasion resistance, fire resistance, water resistance, hygroscopicity, coefficient of friction, biodegradability, plasticity, cushioning, formability, color development, surface adhesion, compressibility, resilience, mycelium species, and chewability of the mycelium formulation required for an application.

13. The method of claim 11,
wherein the attribute information of the mycelium formulation factor is information about at least one of raw material inventory, raw material supply and demand, properties, density, strength, and mycelium species of the mycelium formulation.

14. The method of claim 11,
wherein the attribute information of the mycelium fermentation method factor is information about at least one of raw material inventory, raw material supply and demand, production scale, production rate, mycelium species, the operation status of a production facility, and operation plan of a production facility in the fermentation method.

15. The method of claim 11, further comprising:
a step of determining mycelium fermentation method factor different from a candidate mycelium fermentation method factor based on the attribute information of the factor determined in the step (i) or the attribute information of the factor determined in the step (ii) when it is determined that a production facility corresponding to the candidate mycelium fermentation method factor for determining the mycelium fermentation factor is unable to operate.

16. The method of claim 11,
wherein the step of determining at least one undetermined factor from the factor group based on the attribute information of the factor determined in the step (i) comprises a step of transmitting fifth display information for visualizing and displaying the attribute information to a user terminal.

17. The method of claim 1, further comprising:
a step of transmitting a signal for the mycelium fermentation method factor to a production facility corresponding to the mycelium fermentation method factor.

18-38. (canceled)

39. A non-transitory computer-readable recording medium having recorded a computer program to be executed by a processor,
wherein the computer program is configured to allow the processor to perform the method of claim 1 when the computer program is executed by the processor.

40. An electronic device comprising:
a communication interface configured to communicate with a network;
a processor configured to execute a computer program comprising one or more instructions; and
a memory storing the computer program,
wherein the processor is configured to perform the method of claim 1 when the computer program is executed by the processor.
